(19) United States
(12) Patent Application Publication
(10) Pub. No.: US 2021/0186346 A1
(43) Pub. Date: Jun. 24, 2021

(54) INFORMATION PROCESSING DEVICE, NON-TRANSITORY COMPUTER-READABLE MEDIUM, AND INFORMATION PROCESSING METHOD

(71) Applicant: Mitsubishi Electric Corporation, Tokyo (JP)

(72) Inventors: Yudai NAKAMURA, Tokyo (JP); Masahiro NAITO, Tokyo (JP)

(73) Assignee: Mitsubishi Electric Corporation, Tokyo (JP)

(21) Appl. No.: 17/269,263

(22) PCT Filed: Apr. 24, 2019
(86) PCT No.: PCT/JP2019/017339

§ 371 Date: Feb. 18, 2021

(30) Foreign Application Priority Data
Sep. 10, 2018 (JP) 2018-168453

(51) Int. Cl. A61B 5/021; A61B 5/18

(52) U.S. Cl. CPC A61B 5/02108; A61B 5/18

(57) ABSTRACT

An information processing device includes a skin-region detecting unit, a measurement-region setting unit, a pulse-wave source signal extracting unit, a phase-coincidence-degree calculating unit, and a pulse-wave estimating unit. The skin-region detecting unit detects a skin region of a person in each of multiple frames in a predetermined time period. The measurement-region setting unit sets multiple measurement regions in the skin region. The pulse-wave source signal extracting unit extracts multiple pulse-wave source signals indicating a change in luminance from the multiple measurement regions. The phase-coincidence-degree calculating unit calculates multiple phase coincidence degrees each indicating a degree of phase coincidence between phases of corresponding base components constituting each of the multiple pulse-wave source signals. The pulse-wave estimating unit specifies one of the phase coincidence degrees having the highest degree of phase coincidence and estimates a pulse wave of the person based on the base components corresponding to the specified phase coincidence degree.

### TECHNICAL FIELD

The present invention relates to an information processing device, a program, and an information processing method.

### BACKGROUND ART

It is important to manage and maintain the health of subjects in their daily lives. In particular, the management and maintenance of the health of drivers while they are driving vehicles in their daily lives are especially important for accident prevention. In managing and maintaining the health of drivers, it is effective to constantly acquire biological information, such as heart rate, heart rate variability, respiratory rate, and sweating.

Information on heart rate or heart rate variability, which is included in the biological information, is often used as an index representing the state of autonomic nerves and is important information for managing the health of a driver. In order to directly acquire information regarding heartbeat or heart rate variability, it is necessary to attach electrodes or the like for measuring an electrocardiogram to the chest and measure heart activity; however, this imposes a heavy burden on the driver.

Therefore, instead of directly measuring the activity of the heart, there is a technique of attaching a contact-type device, such as a pulse oximeter, to the fingertip or earlobe and acquiring a pulse wave from a volumetric change in a blood vessel. Even with such a technique, it is necessary to have the device always attached to the fingertip or earlobe, and thus a heavy burden is imposed on the driver, which makes the wearing of such a device unrealistic while driving a vehicle.

As a technique of estimating a pulse wave in a non-contact manner without imposing a burden on a subject, there is, for example, a technique of capturing an image of the face of the subject with a camera and estimating a pulse wave from a small luminance change in the surface of the face of the subject, such as that described in Non-patent Literature 1. In Non-patent Literature 1, multiple measurement regions are set on the face image of the subject, and a frequency power spectrum of the luminance signal acquired in each measurement region is calculated. Pulse waves are combined in accordance with the peak frequencies of the frequency power spectra calculated in the respective regions, and a pulse rate is estimated from the peak of the frequency power spectrum of the combined pulse waves.

### PRIOR ART REFERENCE

### Patent Reference

Non-patent Literature 1: Mayank Kumar, et al., “DistancePPG: Robust non-contact vital signs monitoring using a camera”, Biomedical Optics Express, 6(5), 1565-1588, 2015

### SUMMARY OF THE INVENTION

### Problem to be Solved by the Invention

However, the conventional art has a problem in that the estimation accuracy of a pulse wave decreases when the face of the subject moves. This is because, when the face of the subject moves, a component corresponding to the movement of the face appears as a peak of the frequency power spectrum, and the component corresponding to the movement of the face is erroneously detected as a pulse wave instead of the frequency component corresponding to the pulse wave.

Since it is readily assumed that the face of the driver moves due to the vibration of the vehicle while the vehicle is being driven, it is necessary to accurately estimate the pulse wave even when the face of the subject is moving.

Accordingly, an object of at least one aspect of the present invention is to enable accurate estimation of a pulse wave from frames of video footage even when the face of a person is moving.

### Means of Solving the Problem

An information processing device according to a first aspect of the present invention includes: a skin-region detecting unit configured to detect a skin region in each of multiple frames representing video footage in a predetermined time period, the skin region including skin of a person; a measurement-region setting unit configured to set multiple measurement regions in the skin region; a pulse-wave source signal extracting unit configured to extract, from the multiple measurement regions, multiple pulse-wave source signals corresponding to the respective measurement regions, each of the extracted pulse-wave source signals indicating a change in luminance in the predetermined time period; a phase-coincidence-degree calculating unit configured to calculate multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and a pulse-wave estimating unit configured to specify one of the phase coincidence degrees having the highest degree of phase coincidence of the multiple phase coincidence degrees and estimate a pulse wave of the person based on the base components corresponding to the specified phase coincidence degree.

A program according to an aspect of the present invention causes a computer to function as: a skin-region detecting unit configured to detect a skin region in each of multiple frames representing video footage in a predetermined time period, the skin region including skin of a person; a measurement-region setting unit configured to set multiple measurement regions in the skin region; a pulse-wave source signal extracting unit configured to extract, from the multiple measurement regions, multiple pulse-wave source signals corresponding to the respective measurement regions, each of the extracted pulse-wave source signals indicating a change in luminance in the predetermined time period; a phase-coincidence-degree calculating unit configured to calculate multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and a pulse-wave estimating unit configured to specify one of the phase coincidence degrees having the highest degree of phase coincidence of the multiple phase coincidence degrees and estimate a pulse wave of the person based on the base components corresponding to the specified phase coincidence degree.

An information processing method according to an aspect of the present invention includes the steps of: detecting a skin region in each of multiple frames representing video footage in a predetermined time period, the skin region including skin of a person; setting multiple measurement regions in the skin region; extracting, from the multiple measurement regions, multiple pulse-wave source signals corresponding to the respective measurement regions, each of the extracted pulse-wave source signals indicating a change in luminance in the predetermined time period; calculating multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and specifying one of the phase coincidence degrees having the highest degree of phase coincidence of the multiple phase coincidence degrees and estimate a pulse wave of the person based on the base components corresponding to the specified phase coincidence degree.

### Effects of the Invention

According to at least one aspect of the present invention, a pulse wave can be accurately estimated from a frame of video footage even when the face of a person moves.

### BRIEF DESCRIPTION OF THE DRAWINGS

FIG. 1 is a block diagram that schematically illustrates the configuration of a pulse-wave estimating device according to first, second, and fourth embodiments.

FIGS. 2A to 2C are schematic diagrams illustrating an example of setting measurement regions through facial organ detection.

FIG. 3 is a schematic diagram illustrating a specific setting method of measurement regions.

FIG. 4 is a block diagram schematically illustrating the configuration of a phase-coincidence-degree calculating unit according to the first embodiment.

FIGS. 5A and 5B are schematic diagrams illustrating hardware configuration examples.

FIG. 6 is a flowchart illustrating the operation of the pulse-wave estimating device according to the first embodiment.

FIG. 7 is a schematic diagram illustrating the positional relationship of the face of a subject, an image capturing device, and a light source of ambient light, in the first embodiment.

FIGS. 8A to 8C are schematic diagrams illustrating examples of images acquired by capturing the face of a subject with the image capturing device, in the first embodiment.

FIGS. 9A to 9D are graphs illustrating changes in the average luminance in measurement regions when the face of the subject moves, in the first embodiment.

FIG. 10 is a block diagram schematically illustrating the configuration of a phase-coincidence-degree calculating unit according to the second and fourth embodiments.

FIG. 11 is a block diagram schematically illustrating the configuration of a pulse-wave estimating device according to a third embodiment.

FIG. 12 is a block diagram schematically illustrating the configuration of a phase-coincidence-degree calculating unit according to the third embodiment.

FIG. 13 is a schematic diagram illustrating the positional relationship of the face of a subject, an image capturing device, and a light source of ambient light, in the third embodiment.

FIGS. 14A to 14C are schematic diagrams illustrating examples of images acquired by capturing the face of a subject with an image capturing device, in the third embodiment.

FIGS. 15A to 15F are graphs illustrating changes in the average luminance in measurement regions when the face of the subject moves, in the third embodiment.

### MODE FOR CARRYING OUT THE INVENTION

### First Embodiment

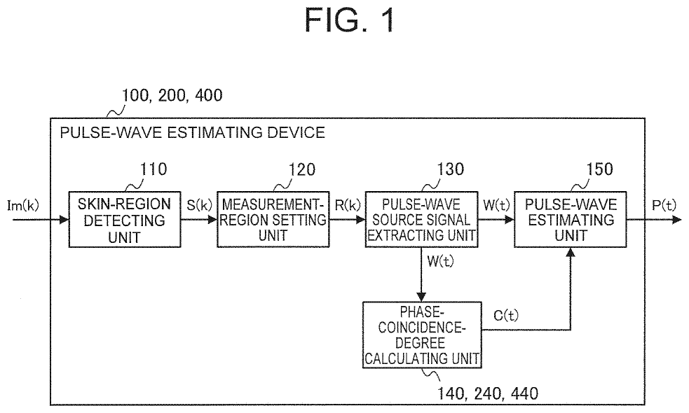

FIG. 1 is a block diagram schematically illustrating the configuration of a pulse-wave estimating device 100 serving as an information processing device according to the first embodiment.

The pulse-wave estimating device 100 is a device that can execute a pulse-wave estimating method, which is an information processing method, according to the first embodiment.

As illustrated in FIG. 1, the pulse-wave estimating device 100 includes a skin-region detecting unit 110, a measurement-region setting unit 120, a pulse-wave source signal extracting unit 130, a phase-coincidence-degree calculating unit 140, and a pulse-wave estimating unit 150.

The outline of the pulse-wave estimating device 100 will now be described. The pulse-wave estimating device 100 receives image data of video footage composed of a series of frames Im(k) representing images of a space including a skin region of a subject captured at a predetermined frame rate Fr. Here, the character “k” denotes the frame number assigned to each frame. For example, the frame provided at the timing immediately after a frame Im(k) is a frame Im(k+1). The pulse-wave estimating device 100 then outputs a pulse wave estimation result P(t) based on a series of frames Im(k-Tp+1) to Im(k) for every specific number of frames Tp. Here, the character “t” denotes the output number assigned for every specific number of frames Tp. For example, the pulse wave estimation result provided at the timing immediately after the pulse wave estimation result P(t) is a pulse wave estimation result P(t+1).

Here, each of the frame number k and the output number t is an integer of one or more. The number of frames Tp is an integer of two or more.

Note that the number of subjects, which is the number of people included in the image data, may be one or more. For simplification purposes, hereinafter, it is assumed that the number of subjects included in the image data is one.

The frame rate Fr is, for example, preferably 30 frames per second. The image data is, for example, a color image, a grayscale image, or a distance image. For simplification purposes, a case in which the image data is an eight-bit grayscale image having a width of 640 pixels and a height of 480 pixels will be described below. The number of frames Tp may be any value; for example, the number of frames Tp may correspond to the number of the frames in 10 seconds; and for the example above, 300 frames are preferable.

The components constituting the pulse-wave estimating device 100 will now be described.

The skin-region detecting unit 110 detects a skin region that includes the skin of the subject in the frames Im(k) included in the image data provided as input information from the image capturing device described below serving as the image capturing unit, and generates skin region information S(k) indicating the detected skin region. The generated skin region information S(k) is provided to the measurement-region setting unit 120.

The skin region according to the first embodiment will be described as a region corresponding to the face of the subject. However, the skin region may be any region different from the face of the subject. For example, the skin region may be a region corresponding to a section of the face, such as the eye, eyebrow, nose, mouth, forehead, cheek, or chin. Alternatively, the skin region may be a region corresponding to a body section different from the face, such as the head, shoulder, wrist, neck, or foot. Note that the skin region may include multiple regions.

The skin region information S(k) can include information indicating whether or not a skin region is detected and information indicating the position and size of the detected skin region on the image. Here, the skin region information S(k) will be described as information indicating a rectangular region representing the position and size of the face on the image.

Specifically, when the skin region corresponds to the face of the subject, the skin region information S(k) indicates, for example, whether or not the face of the subject is detected, the center coordinates Fc (Fcx, Fcy) of a rectangle surrounding the face, and the width Fcw and the height Fch of the rectangle.

For whether or not the face is detected, for example, when the face is detected, the value “1” is provided, and when the face is not detected, the value “0” is provided.

The center coordinates of the rectangle are represented in a coordinate system of the frames Im(k), where the upper left corner of the frames Im(k) is defined as the origin, the rightward direction of the frames Im(k) is defined as the positive direction along the x-axis, and the downward direction of the frames Im(k) is defined as the positive direction along the y-axis.

The detection of the face of the subject can be implemented by using a known means. For example, a cascade-type face detector using Haar-like features can be used to extract a rectangular region surrounding the face of the subject.

The measurement-region setting unit 120 receives the frames Im(k) and the skin region information S(k), sets multiple measurement regions for extracting pulse wave signals in an image region corresponding to the skin region indicated by the skin region information S(k), and generates measurement region information R(k) indicating the set measurement regions. The generated measurement region information R(k) is provided to the pulse-wave source signal extracting unit 130.

The measurement region information R(k) can include information indicating the positions and sizes of the Rn (positive integer) measurement regions on an image. Each of the measurement regions is referred to as a measurement region ri(k) (where i=1, 2 . . . , Rn). Here, a measurement region ri(k) will be described as having a shape of a quadrilateral, and the position and the size of the measurement region ri(k) are defined by the four vertices of the quadrilateral on the image.

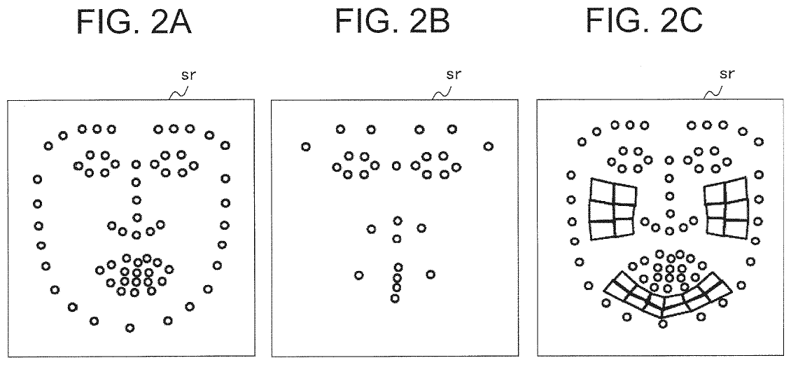

As an example of a setting method of measurement regions ri(k), an example using facial organ detection will now be described with reference to FIG. 2.

The measurement-region setting unit 120 first detects Ln (positive integer) landmarks of facial organs (outer corners of eyes, inner corners of eyes, nose, mouth, etc.), such as those illustrated in FIG. 2A or 2B, in a skin region sr indicated by the skin region information S(k) and defines a vector L(k) that stores the coordinate values of the respective landmarks. In FIGS. 2A and 2B, the landmarks are indicated as circles.

The facial organ detection can be implemented by using a known means. For example, the coordinates of the landmarks of facial organs can be detected using a model known as a constrained local model (CLM). Although not particularly limited, the number Ln of the landmarks is preferably 66, as illustrated in FIG. 2A, or 29, as illustrated in FIG. 2B. It is desirable to determine the number of landmarks in accordance with the hardware, such as the CPU, because a large number of landmarks leads to stable detection results but also causes an increase in the amount of processing. In the description below, the number Ln of landmarks is 66.

The measurement-region setting unit 120 then sets the vertex coordinates of the quadrilateral of each measurement region ri(k) with reference to the detected landmarks. For example, the measurement-region setting unit 120 sets the vertex coordinates of quadrilaterals as illustrated in FIG. 2C to set Rn measurement regions ri(k). Here, for example, the number of measurement regions Rn is 12.

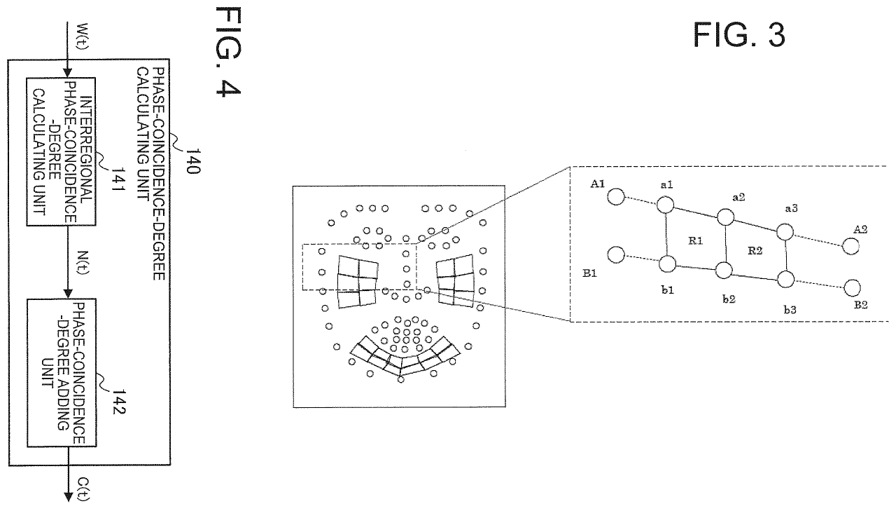

A specific setting method of the measurement regions ri(k) will be described with reference to FIG. 3.

Here, a case in which measurement regions ri(k) are set in a section corresponding to a cheek in the skin region sr will be described.

The measurement-region setting unit 120 first selects a landmark A1 on the contour of the face and a landmark A2 on the nose. For example, the measurement-region setting unit 120 may first select the landmark A2 on the nose and select the landmark A1 on the contour of the face closest to the landmark A2 on the nose.

The measurement-region setting unit 120 then sets auxiliary landmarks a1, a2, and a3 such that a segment extending between the landmark A1 and the landmark A2 is equally divided into four sections.

Similarly, the measurement-region setting unit 120 selects a landmark B1 on the contour of the face and a landmark B2 on the nose and sets auxiliary landmarks b1, b2, and b3 that equally divide a segment extending between the landmark B1 and the landmark B2 into four sections. Note that the landmark B1 should be selected from, for example, landmarks on the nose adjacent to the landmark A1. The landmark B2 should be selected from landmarks on the face adjacent to the landmark A2.

The measurement-region setting unit 120 then defines the quadrilateral region surrounded by the auxiliary landmarks a1, b1, b2, and a2 as a measurement region R1. The auxiliary landmarks a1, b1, b2, and a2 are the vertex coordinates corresponding to the measurement region R1.

Similarly, the measurement-region setting unit 120 defines the quadrilateral region surrounded by the auxiliary landmarks a2, b2, b3, and a3 as a measurement region R2. The auxiliary landmarks a2, b2, b3, and a3 are the vertex coordinates corresponding to the measurement region R2.

The measurement-region setting unit 120 performs similar processes to sections corresponding to the other cheek and the chin, to set the vertex coordinates of the quadrilaterals of the measurement regions ri(k).

The measurement-region setting unit 120 then generates information including the coordinates of the four vertices of the respective measurement regions ri(k) as measurement region information R(k) and provides the measurement region information R(k) to the pulse-wave source signal extracting unit 130.

Note that, in the example described above, the measurement-region setting unit 120 detects the coordinates of the landmarks with a CLM, but detection of the landmarks is not limited thereto. For example, the measurement-region setting unit 120 may use a tracking technique, such as a Kanade-Lucas-Tomasi (KLT) tracker. Specifically, the measurement-region setting unit 120 may detect the coordinates of landmarks in the first frame Im(1) with a CLM, track the landmark coordinates in the second frame Im(2) and the subsequent frames with a KLT tracker, and calculate the landmark coordinates for each frame Im(k). By performing tracking, a CLM does not have to be implemented to the respective frames Im(k), and thereby the amount of processing can be reduced. Since, in such a case, detection errors of the tracking accumulate, the measurement-region setting unit 120 may implement a CLM every several frames and perform a reset process for resetting the coordinate positions of the landmarks.

Note that the positions of the measurement regions are not limited to those of the 12 regions illustrated in FIG. 2C. For example, the forehead section and/or the tip of the nose may be included. Furthermore, the measurement-region setting unit 120 may change the set regions depending on the subject. For example, for a subject who has bangs hanging over the forehead, the measurement-region setting unit 120 may detect the bangs and exclude the forehead region from the measurement regions. For a subject who is wearing glasses with a thick frame, the measurement-region setting unit 120 may detect the position of the glasses and exclude the corresponding region from the measurement regions. Moreover, for a subject who has a beard, the measurement-region setting unit 120 may exclude the beard region from the measurement regions. A measurement region may overlap with another measurement region.

Referring back to FIG. 1, the pulse-wave source signal extracting unit 130 receives the frames Im(k) and the measurement region information R(k), extracts pulse-wave source signals each of which indicates a change in the luminance during a time period corresponding to the number of frames Tp included in a predetermined time period from each of the measurement regions ri(k) indicated by the measurement region information R(k) and generates pulse-wave source signal information W(t) indicating the extracted pulse-wave source signals. Note that the pulse-wave source signals are source signals of a pulse wave. The generated pulse-wave source signal information W(t) is provided to the phase-coincidence-degree calculating unit 140 and the pulse-wave estimating unit 150.

The pulse-wave source signal information W(t) can include information indicating a pulse-wave source signal wi(t) extracted from a measurement region ri(k). The pulse-wave source signal wi(t) is time-series data of Tp frames and is extracted on the basis of, for example, Tp frames Im(k−Tp+1), Im(k−Tp+2) . . . , Im(k) of the past and measurement region information R(k−Tp+1), R(k−Tp+2) . . . , R(k).

For the extraction, the pulse-wave source signal extracting unit 130 calculates luminance feature amounts Gi(j) (j=k−Tp+1, k−Tp+2 . . . , k) of the respective measurement regions ri(k) for the respective frames Im(k). The luminance feature amounts Gi(j) are values calculated on the basis of luminance values in the respective measurement regions ri(j) in the respective frames Im(j) and, for example, are each an average or variance of the luminance values of pixels included in each of the measurement regions ri(j). Here, the luminance feature amount Gi(j) will be described as an average of the luminance values of pixels included in each measurement region ri(j). The luminance feature amounts Gi(j) calculated for the respective frames Im(k) and chronologically arranged are defined as a pulse-wave source signal wi(t). That is, pulse-wave source signal wi(t)=[Gi(k−Tp+1), Gi(k−Tp+2) . . . , Gi(k)].

The pulse-wave source signal extracting unit 130 then generates pulse-wave source signal information W(t) by putting together the pulse-wave source signals wi(t) of the respective measurement regions ri(k). The generated pulse-wave source signal information W(t) is provided to the phase-coincidence-degree calculating unit 140 and the pulse-wave estimating unit 150.

Note that the pulse-wave source signals wi(t) include various noise components in addition to the above-described pulse-wave components and facial movement components. An example of a noise component is noise due to an element defect of an image capturing device as described below. In order to remove such noise components, it is desirable to perform a filtering process as preprocessing of the pulse-wave source signals wi(t).

In the filtering process, the pulse-wave source signals wi(t) are processed by using, for example, a low-pass filter, a high-pass filter, or a band-pass filter. In the following description, it is assumed that a bandpass filter is used.

As the band-pass filter, for example, a Butterworth filter or the like can be used. As the cutoff frequency of a band-pass filter, it is desirable that, for example, the lower cutoff frequency be 0.5 Hz and the higher cutoff frequency be 5.0 Hz.

Note that the type of filtering is not limited to the above-described Butterworth filter. The cut-off frequency is also not limited to the above-mentioned frequencies. The type of filtering and the cut-off frequency should be set in accordance with a condition or a circumstance of the subject.

The phase-coincidence-degree calculating unit 140 receives the pulse-wave source signal information W(t), calculates phase coincidence degrees each indicating the degree of phase coincidence of the phases of multiple base components corresponding to each other in the pulse-wave source signal information W(t), and generates phase coincidence degree information C(t) indicating the degrees of phase coincidence of the respective base components. The phase coincidence degree information C(t) is provided to the pulse-wave estimating unit 150. Since the phase is an attribute of the base component, it can be said that the degree of phase coincidence is the degree of attribute coincidence, and the phase coincidence degree is the attribute coincidence degree. Therefore, the phase-coincidence-degree calculating unit 140 can also be regarded as an attribute-coincidence-degree calculation unit.

Specifically, the phase-coincidence-degree calculating unit 140 selects a pair consisting of two pulse-wave source signals from the multiple pulse-wave source signals indicated by the pulse-wave source signal information W(t). Here, the two pulse-wave source signals of the selected pair are referred to as a first pulse-wave source signal and a second pulse-wave source signal. The phase-coincidence-degree calculating unit 140 calculates, between the respective base components constituting the first pulse-wave source signal and the respective base components constituting the second pulse-wave source signal, multiple coincidence degrees indicating the degrees of phase coincidence of corresponding base components. The phase-coincidence-degree calculating unit 140 then specifies multiple attribute coincidence degrees in accordance with the calculated coincidence degrees. Here, the respective attribute coincidence degrees correspond to the respective base components.

Note that the phase-coincidence-degree calculation unit 140 may select one pair or two or more pairs.

When multiple pairs are selected, the phase-coincidence-degree calculating unit 140 may add, for respective corresponding base components, the multiple coincidence degrees calculated for the multiple pairs and specify the multiple added values as multiple phase coincidence degrees.

When one pair is selected, the phase-coincidence-degree calculating unit 140 may specify the multiple coincidence degrees calculated for the pair as multiple phase coincidence degrees.

In the following description, it is assumed that the phase-coincidence-degree calculating unit 140 selects multiple pairs.

FIG. 4 is a block diagram schematically illustrating the configuration of the phase-coincidence-degree calculating unit 140.

The phase-coincidence-degree calculating unit 140 includes an interregional phase-coincidence-degree calculating unit 141 and a phase-coincidence-degree adding unit 142.

The interregional phase-coincidence-degree calculating unit 141 selects, on the basis of the pulse-wave source signal information W(t), a pair consisting of two measurement regions ru(k) and rv(k) from the multiple measurement regions ri(k) used for the calculation of the pulse-wave source signals wi(t), and selects a pair consisting of two pulse-wave source signals wu(t) and wv(t) corresponding to the pair consisting of the two measurement regions ru(k) and rv(k). The interregional phase-coincidence-degree calculating unit 141 calculates an interregional phase coincidence degree cuv(t) that is the degree of phase coincidence of the phases of the base components of the two pulse-wave source signals wu(t) and wv(t) of the selected pair. Note that it is assumed that u, v=1, 2 . . . , Rn, where u≠v.

Note that at least one such pair should be generated; alternatively, multiple pairs may be generated to calculate multiple interregional phase coincidence degrees cuv(t). The interregional phase-coincidence-degree calculating unit 141 provides interregional phase-coincidence-degree information generated by putting together the generated interregional phase coincidence degrees cuv (t), to the phase-coincidence-degree adding unit 142.

The operation of calculating the degree of phase coincidence of base components performed by the interregional phase-coincidence-degree calculating unit 141 will now be described in detail.

In calculating the degree of phase coincidence of base components, first, the interregional phase-coincidence-degree calculating unit 141 decomposes a pulse-wave source signal wi(t) into base components. A case in which frequency components are used as the base components will be described as an example below. Note that the base components are the signal components constituting a pulse-wave source signal wi(t) and are signal components that can express a pulse-source signal when the base components are given as arguments of a certain function.

First, the interregional phase-coincidence-degree calculating unit 141 decomposes the pulse-wave source signal wi(t) of each measurement region ri(k) included in the pulse-wave source signal information W(t) into frequency components. In order to decompose the pulse-wave source signal wi(t) into frequency components, for example, fast Fourier transform (FFT) is used. FFT can decompose the pulse-wave source signal wi(t), which is time-series data, into data of frequency components (the magnitudes (powers) and phases of the respective frequency components). The magnitude of each of the frequency components f when FFT is performed on the pulse-wave source signal wi(t), which is time-series data, is defined as |Fi(f, t)|, and the phase is defined as ∠Fi(f, t). Note that, when FFT is performed, f=0, Δf, 2×Δf . . . , SrxΔf/2 holds because aliasing occurs around the Nyquist frequency (half of the sampling frequency). Here, Δf is a value determined by Tp frames, which is the length of the time-series data, and Δf=1/Ts when Tp frames=Ts seconds.

The interregional phase-coincidence-degree calculating unit 141 then selects a pair consisting of two measurement regions ru(k) and rv(k) from the multiple measurement regions ri(k) used for the calculation of the pulse-wave source signal wi(t) and calculates an interregional phase coincidence degree cuv(t) that is the degree of phase coincidence of the base components of the two measurement regions ru(k) and rv(k) of the pair. The interregional phase coincidence degree cuv(t) is calculated, for example, as an absolute value of the difference between a phase ∠Fu(f, t) of a measurement region u and a phase ∠Fv(f, t) of a measurement region v. By obtaining the absolute value of the phase difference for each frequency component, the degrees of phase coincidence of the respective frequency components can be calculated.

Note that, in such a case, the smaller the absolute value of the phase difference, the higher the degree of phase coincidence, and the larger the absolute value of the phase difference, the lower the degree of phase coincidence. The degree of phase coincidence for every frequency component is calculated, and the calculated degrees are arranged and referred to as an interregional phase coincidence degree cuv(t).

The interregional phase-coincidence-degree calculating unit 141 then generates interregional phase-coincidence-degree information N(t) by putting together the interregional phase coincidence degree cuv(t) of respective frequency components.

Note that the interregional phase-coincidence-degree calculating unit 141 may not calculate the degree of phase coincidence for all frequency components, but may calculate the degree of phase coincidence only for the frequency components satisfying a specific condition. For example, when the power (magnitude) of a frequency component is significantly small, the frequency component can be regarded as a noise component rather than a pulse wave component; therefore, the interregional phase-coincidence-degree calculating unit 141 does not calculate the degree of phase coincidence for the corresponding frequency component. Alternatively, the interregional phase-coincidence-degree calculating unit 141 may assign a constant to the degree of phase coincidence of the corresponding frequency component, assuming that the degree of phase coincidence is quasi-low.

Given the interregional phase-coincidence-degree information N(t), the phase-coincidence-degree adding unit 142 adds the interregional phase coincidence degrees cuv(t) for every base component and generates phase coincidence degree information C(t) indicating the phase coincidence degrees for the respective base components between the measurement regions ri(k). The phase coincidence degree information C(t) is provided to the pulse-wave estimating unit 150. The phase coincidence degree information C(t) is calculated, for example, by adding the interregional phase coincidence degrees cuv(t) included in the interregional phase-coincidence-degree information N(t) for every frequency component.

The calculation method of the phase coincidence degree information C(t) is not limited to addition for every component; alternatively, multiplication or the like may be used.

When the interregional phase-coincidence-degree calculating unit 141 selects only one pair consisting of two measurement regions ru(k) and rv(k) from the measurement regions ri(k) used for the calculation of the pulse-wave source signal wi(t), the phase-coincidence-degree adding unit 142 does not have to be provided, and the phase-coincidence-degree adding unit 142 may provide the interregional phase-coincidence-degree information N(t) to the pulse-wave estimating unit 150 as phase coincidence degree information C(t).

Referring back to FIG. 1, the pulse-wave estimating unit 150 estimates a pulse wave on the basis of the pulse-wave source signal information W(t) and the phase coincidence degree information C(t) and outputs a pulse-wave estimation result P(t) that is pulse wave information indicating the estimated pulse wave. Pulse wave information may be, for example, time-series data of an estimated pulse wave or pulse rate. For simplification purposes, the pulse wave information is assumed to indicate pulse rate (pulses per minute).

For example, when the pulse rate is output as the pulse-wave estimation result P(t), the pulse-wave estimating unit 150 specifies the frequency component that is the base component having the highest degree of phase coincidence in the phase coincidence degree information C(t) for every frequency component and estimates the pulse wave on the basis of the specified frequency component. Specifically, the pulse-wave estimating unit 150 assumes that the frequency component having the highest degree of phase coincidence corresponds to the pulse wave, and outputs the frequency of the frequency component corresponding to the pulse wave as the pulse rate.

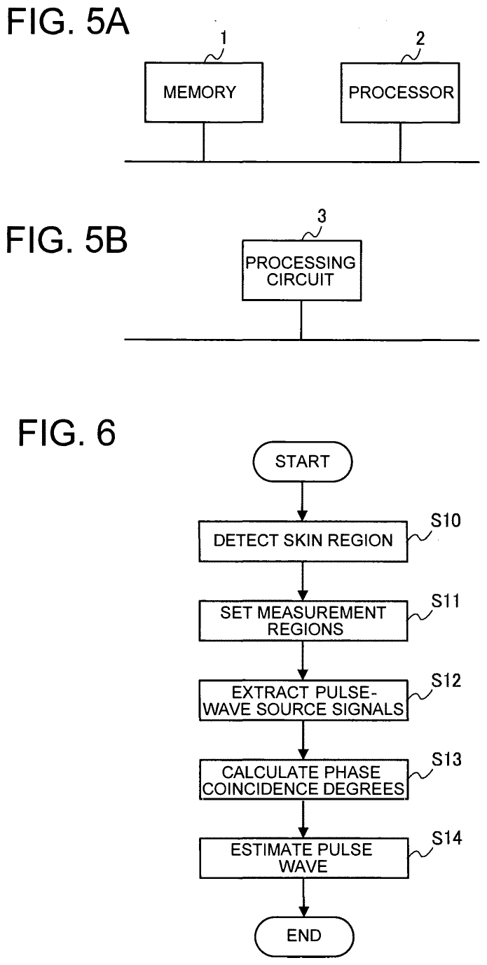

A portion or the entirety of the skin-region detecting unit 110, the measurement-region setting unit 120, the pulse-wave source signal extracting unit 130, the phase-coincidence-degree calculating unit 140, and the pulse-wave estimating unit 150 described above can be implemented by, for example, a memory 1 and a processor 2, such as a central processing unit (CPU), that executes programs stored in the memory 1, as illustrated in FIG. 5A. Such programs may be provided via a network or may be recorded and provided on a recording medium. That is, such programs may be provided as, for example, program products.

Furthermore, a portion or the entirety of the skin-region detecting unit 110, the measurement-region setting unit 120, the pulse-wave source signal extracting unit 130, the phase-coincidence-degree calculating unit 140, and the pulse-wave estimating unit 150 can be implemented by, for example, a processing circuit 3, such as a single circuit, a composite circuit, a programmed processor, a parallel programmed processor, an application-specific integrated circuit (ASIC), or a field programmable gate array (FPGA), as illustrated in FIG. 5B.

FIG. 6 is a flowchart illustrating the operation of the pulse-wave estimating device 100 according to the first embodiment.

The operation illustrated in FIG. 6 is performed for every frame to which a captured image is input, that is, the operation is performed once every frame period.

The skin-region detecting unit 110 first detects a skin region of the subject from the frames Im(k) provided as input information from the image capturing device described below and generates skin region information S(k) indicating the detected skin region (step S10). The generated skin region information S(k) is provided to the measurement-region setting unit 120.

The measurement-region setting unit 120 then receives the frames Im(k) and the skin region information S(k), sets multiple measurement regions ri(k) for extracting pulse wave signals from the skin region indicated by the skin region information S(k), and generates measurement region information R(k) indicating the set measurement regions ri(k) (step S11). The generated measurement region information R(k) is provided to the pulse-wave source signal extracting unit 130.

The pulse-wave source signal extracting unit 130 receives the frames Im (k) and the measurement region information R(k), extracts the pulse-wave source signal wi(t) serving as the source of a pulse wave on the basis of the luminance values in the respective measurement regions ri(k) indicated by the measurement region information R(k), and generates pulse-wave source signal information W(t) indicating the extracted pulse-wave source signal wi(t) (step S12). The generated pulse-wave source signal information W(t) is provided to the phase-coincidence-degree calculating unit 140 and the pulse-wave estimating unit 150.

The phase-coincidence-degree calculating unit 140 then receives the pulse-wave source signal information W(t), calculates the degree of phase coincidence between the measurement regions ri(k) for the base components included in the pulse-wave source signal wi(t) indicated by the pulse-wave source signal information W(t), and generates phase coincidence degree information C(t) indicating the degree of phase coincidence for every base component (step S13). The phase coincidence degree information C(t) is provided to the pulse-wave estimating unit 150.

The pulse-wave estimating unit 150 then estimates a pulse wave on the basis of the pulse-wave source signal information W(t) and the phase coincidence degree information C(t) and outputs a pulse-wave estimation result P(t) indicating the estimated pulse wave (step S14).

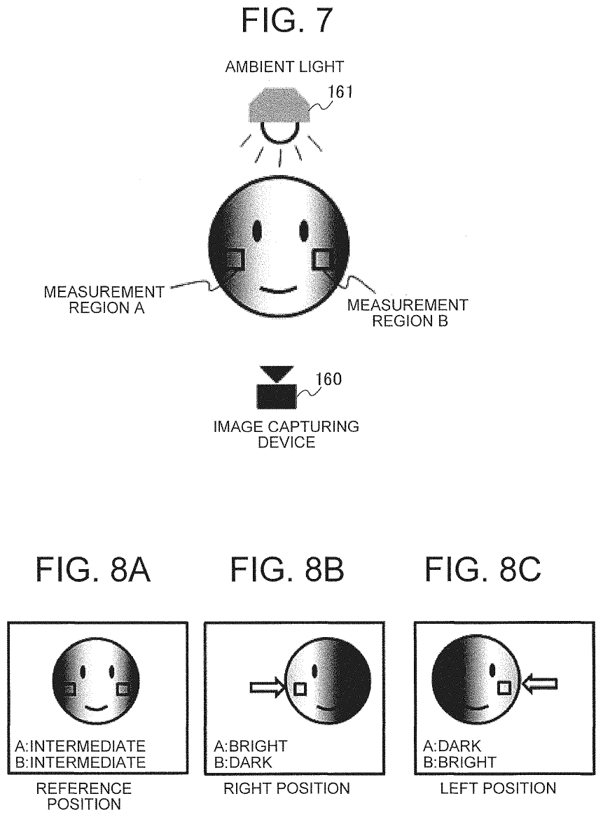

The effects of the pulse-wave estimating device 100 according to the first embodiment will now be described with reference to FIGS. 7 to 9.

FIG. 7 is a schematic diagram illustrating the positional relationship of the face of the subject, an image capturing device 160, and a light source 161 of ambient light.

As illustrated in FIG. 7, it is assumed that a measurement region A and a measurement region B are disposed in the skin region of the face of the subject.

FIGS. 8A to 8C illustrate examples of images obtained by capturing the face of the subject by the image capturing device 160 illustrated in FIG. 7.

The image illustrated in FIG. 8A is an example in which the face of the subject is positioned at the center of the image capturing device 160. Such a position is referred to as a reference position.

The image illustrated in FIG. 8B is an example in which the face of the subject is positioned to the right of the center of the image capturing device 160. Such a position is referred to as a right position.

The image illustrated in FIG. 8C is an example in which the face of the subject is positioned to the left of the center of the image capturing device 160. Such a position is referred to as a left position.

When the face is positioned at the right position, the measurement region A is brighter than when the face is positioned at the reference position; and when the face is positioned at the left position, the measurement region A is darker than when the face is positioned at the reference position.

When the face is positioned at the right position, the measurement region B is darker than when the face is positioned at the reference position; and when the face is positioned at the left position, the measurement region B is brighter than when the face is positioned at the reference position.

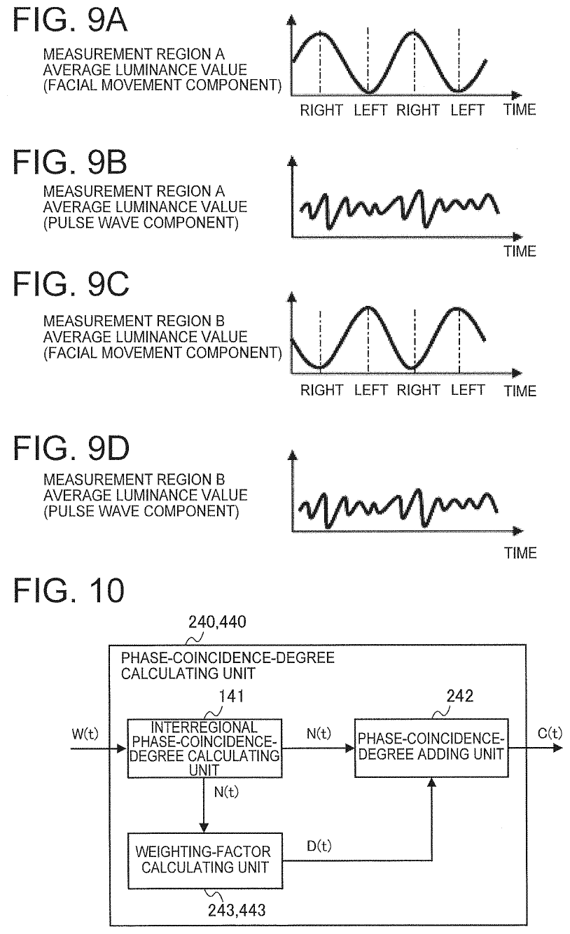

FIGS. 9A to 9D illustrate changes in average luminance values in the measurement regions A and B when the face of the subject moves in the order of the reference position, the right position, the reference position, the left position, the reference position, the right position, the reference position, and the left position.

FIG. 9A illustrates a change in the average luminance value of the facial movement components in the measurement region A when the face of the subject moves as described above; and FIG. 9B illustrates a change in the average luminance value of the pulse wave components of the face in the measurement region A when the face of the subject moves as described above.

FIG. 9C illustrates a change in the average luminance value of the facial movement components of the face in the measurement region B when the face of the subject moves as described above; and FIG. 9D illustrates a change in the average luminance value of the pulse wave components of the face in the measurement region B when the face of the subject moves as described above.

As illustrated in FIGS. 9A to 9D, when the face of the subject moves, the average luminance values in the measurement regions change in response. At this time, the frequencies of the facial movement components due to the luminance value variation may be similar values depending on the measurement regions, but the phases are different.

For example, as illustrated in FIGS. 9A and 9C, the timings of brightening and darkening are different between the measurement region A and the measurement region B; in other words, since the measurement region B is dark when the measurement region A is bright, the phases of the respective frequency components are different when the luminance value change is viewed as a signal component.

As illustrated in FIGS. 9B and 9D, the frequencies and phases of the pulse wave components due to the luminance value change are similar values in all measurement regions.

That is, since the luminance value change due to facial movement causes a phase difference whereas the luminance value change due to a pulse wave result in similar values, the degrees of phase coincidence of the base components of the respective measurement regions can be compared to discriminate between components of the luminance value change due to facial movement and components of the luminance value change due to a pulse wave.

In particular, by selecting a base component having a high degree of phase coincidence as the pulse wave component, the influence of the facial movement can be suppressed, and the pulse wave can be estimated with high accuracy.

Since the luminance value change in the measurement regions has the above-described characteristics, the pulse-wave estimating device 100 according to the first embodiment can estimate a pulse wave on the basis of the degree of phase coincidence of base components of extracted pulse-wave source signals between the measurement regions, and thereby suppress a decrease in accuracy due to facial movement and estimate a pulse wave with high accuracy.

By generating pairs each consisting of two regions selected from multiple measurement regions and calculating and adding the coincidence degrees of the base components of the respective pairs, the degrees of phase coincidence of the base components between the multiple measurement regions can be calculated, and thereby the pulse wave can be estimated with higher accuracy.

In the first embodiment, the image data is described as a grayscale image, but the image data is not limited thereto. For example, an RGB image may be used as image data. Furthermore, the grayscale image described above may be image data obtained by an image capturing device capable of receiving near-infrared light (for example, light having a wavelength of 850 nm, 940 nm, etc.). In such a case, the pulse-wave estimating device 100 according to the first embodiment can estimate a pulse wave even at night by illuminating the subject and capturing an image of the subject with an illumination device of near-infrared light used.

Note that, in the first embodiment, it is assumed that the ambient light is emitted from above, as illustrated in FIG. 7, but the emission is not limited thereto. For example, the ambient light may be emitted from one side.

Note that, in the first embodiment, the pulse-wave estimation result P(t) is assumed to be a pulse rate, but the pulse-wave estimation result P(t) is not limited thereto. The pulse-wave estimating unit 150 may assume that, for example, the component having the highest degree of phase coincidence out of the phase coincidence degree information C(t) for every component corresponds to a pulse wave and may perform inverse Fourier transform using the data of the corresponding frequency component to synthesize a pulse wave.

Note that, in the first embodiment, the number of subjects included in the image data is one, but the number of subjects is not limited thereto. In the case of two or more subjects, a pulse wave may be estimated for each subject.

### Second Embodiment

As illustrated in FIG. 1, a pulse-wave estimating device 200 according to the second embodiment includes a skin-region detecting unit 110, a measurement-region setting unit 120, a pulse-wave source signal extracting unit 130, a phase-coincidence-degree calculating unit 240, and a pulse-wave estimating unit 150.

The skin-region detecting unit 110, the measurement-region setting unit 120, the pulse-wave source signal extracting unit 130, and the pulse-wave estimating unit 150 of the pulse-wave estimating device 200 according to the second embodiment are similar to the skin-region detecting unit 110, the measurement-region setting unit 120, the pulse-wave source signal extracting unit 130, and the pulse-wave estimating unit 150, respectively, of the pulse-wave estimating device 100 according to the first embodiment.

Note that the pulse-wave estimating device 200 is a device that can execute a pulse-wave estimating method that is an information processing method according to the second embodiment.

The phase-coincidence-degree calculating unit 240 according to the second embodiment selects multiple pairs each consisting of two pulse-wave source signals from multiple pulse-wave source signals indicated by pulse-wave source signal information W(t). The phase-coincidence-degree calculating unit 240 then calculates multiple coincidence degrees each indicating the degree of phase coincidence between corresponding base components in the respective selected pairs, as in the first embodiment. The phase-coincidence-degree calculating unit 240 then sets weighting factors to the respective pairs, weights the multiple coincidence degrees calculated for the respective pairs by using the respective weighting factors, and specifies the sums of the weighted values of respective corresponding base components as multiple degrees of phase coincidence.

Here, the phase-coincidence-degree calculating unit 240 sets the weighting factors so that the weights are heavier when the multiple coincidence degrees calculated from the respective pairs include those having higher degrees of phase coincidence.

The phase-coincidence-degree calculating unit 240 can also set the weighting factors so that the weights are heavier when the distances between the two measurement regions ru(k) and rv(k) corresponding to the respective pairs are larger.

FIG. 10 is a block diagram schematically illustrating the configuration of the phase-coincidence-degree calculating unit 240 according to the second embodiment.

The phase-coincidence-degree calculating unit 240 includes an interregional phase-coincidence-degree calculating unit 141, a phase-coincidence-degree adding unit 242, and a weighting-factor calculating unit 243.

The interregional phase-coincidence-degree calculating unit 141 of the phase-coincidence-degree calculating unit 240 according to the second embodiment is similar to the interregional phase-coincidence-degree calculating unit 141 of the phase-coincidence-degree calculating unit 140 according to the first embodiment. However, the interregional phase-coincidence-degree calculating unit 141 according to the second embodiment provides interregional phase-coincidence-degree information N(t) to the phase-coincidence-degree adding unit 242 and the weighting-factor calculating unit 243.

The weighting-factor calculating unit 243 receives the interregional phase-coincidence-degree information N(t), calculates weighting factors duv(t) for the respective interregional phase coincidence degrees cuv(t) indicated by the interregional phase-coincidence-degree information N(t), and generates weighting information D(t) indicating the calculated weighting factors duv(t). The weighting information D(t) is provided to the phase-coincidence-degree adding unit 242.

The weighting information D(t) can include the weighting factors duv(t) for the respective interregional phase coincidence degrees cuv(t) included in the interregional phase-coincidence-degree information N(t). A weighting factor duv(t) is, for example, a value between “0” and “1.” For example, when the weighting factor duv(t) is “0,” the weight for the corresponding interregional phase coincidence degree cuv(t) is small, and when the weighting factor duv(t) is “1,” the weight for the corresponding interregional phase coincidence degree cuv(t) is large. When the weighting factor duv(t) is “0.5”, the weight is an intermediate value between those of “0” and “1.”

The weighting factor duv(t) is determined on the basis of, for example, the interregional phase coincidence degree cuv(t). As described above, when the absolute value of the phase difference of the base components acquired in each measurement region ri(k) is used as the interregional phase coincidence degree cuv(t), the higher the coincidence between the phases of the base components of the two measurement regions ru(k) and rv(k), the smaller each element of the interregional phase coincidence degree cuv(t). Therefore, when the interregional phase coincidence degrees cuv(t) include more small values, there are more base components having phases coinciding more between the two measurement regions ru(k) and rv(k). That is, the more small values are included in the interregional phase coincidence degree cuv(t), the larger the corresponding weighting factor duv(t) is set; in this way, the degree of phase coincidence between the measurement regions ri(k) can be calculated by using the information of the pair consisting of the two measurement regions ru(k) and rv(k) and including pulse wave components.

Based on the above, in order to set the weight larger as the phases coincide more, it is desirable to determine the corresponding weighting factor duv(t), for example, on the basis of the minimum value of the interregional phase coincidence degree cuv(t).

In the following description, a method using the minimum value of the interregional phase coincidence degree cuv(t) will be described as a method of determining the weighting factor duv(t).

The maximum value and the minimum value that can be taken by the interregional phase coincidence degree cuv(t) are cₘₐₓ and cₘᵢₙ, respectively. When the minimum value of the corresponding interregional phase coincidence degree cuv(t) is cuvₘᵢₙ (t), the weighting factor duv(t) is calculated by the following equation (1).

duv(t)=1.0−(cuvₘᵢₙ(t)−cₘᵢₙ)/(cₘₐₓ−cₘᵢₙ) (1)

By calculating the weighting factor duv(t) by using the above equation, the higher the coincidence of the phases, i.e., the smaller the values included in the corresponding interregional phase coincidence degree cuv(t), the larger the weighting factor duv(t) can be set.

The weighting-factor calculating unit 243 puts together the weighting factors duv(t) calculated for the respective interregional phase coincidence degrees cuv(t) and generates weighting information D(t) indicating the weighting factors duv(t). The weighting information D(t) is provided to the phase-coincidence-degree adding unit 242.

The phase-coincidence-degree adding unit 242 receives the interregional phase-coincidence-degree information N(t) and the weight information D(t), generates phase coincidence degree information C(t) for every base component between the measurement regions and provides the generated phase coincidence degree information C(t) to the pulse-wave estimating unit 150.

Specifically, the phase-coincidence-degree adding unit 242 weights the interregional phase coincidence degrees cuv(t) indicated by the interregional phase-coincidence-degree information N(t) for the respective base components by using the corresponding weighting factors duv(t) indicated by the weight information D(t), and adds the weighted values.

The weighting addition is performed as shown by the following equation (2).

Σu,v(duv(t)×cuv(t)) (2)

As defined by equation (2), the phase-coincidence-degree adding unit 242 multiplies the interregional phase coincidence degree cuv(t) indicated by the interregional phase-coincidence-degree information N(t) by the corresponding weight information D(t) and adds the results for every base component, to generate phase coincidence degree information C(t) indicating the degrees of phase coincidence for the respective base components between the measurement regions ri(k). The phase coincidence degree information C(t) is provided to the pulse-wave estimating unit 150.

As described above, with the pulse-wave estimating device 200 according to the second embodiment, the weighting factor duv(t) is calculated on the basis of the minimum value of the interregional phase coincidence degree cuv(t), or in other words, the weighting factor duv(t) is calculated so that the smaller the minimum value of the interregional phase coincidence degree cuv(t), the larger the weighting factor duv (t); in this way, the degree of phase coincidence between the measurement regions ri(k) can be calculated by placing more emphasis on pairs of the measurement regions ri(k) having phases that are more aligned. Therefore, the second embodiment can estimate the pulse wave with higher accuracy.

Note that, in the second embodiment, the weighting factor duv(t) is calculated on the basis of the minimum value of the interregional phase coincidence degree cuv(t), but the calculation method of the weighting factor duv(t) is not limited thereto. For example, a corresponding weighting factor duv(t) may be determined in accordance with the distance between two measurement regions ru(k) and rv(k). As for the facial movement component included in the change in the average luminance value, a phase difference is more likely to occur if the distance between the two regions used for the calculation of the interregional phase coincidence degree cuv(t) is large. Therefore, the weighting-factor calculating unit 243 calculates the distance between the two measurement regions ru(k) and rv(k) on the basis of the positions of the measurement regions ri(k) on the image and sets the weighting factor duv(t) in accordance with the calculated distance. At this time, the weighting-factor calculating unit 243 sets the weighting factor duv(t) so that the larger the distance is, the larger the value is.

The weighting factor duv(t) may be calculated using a method other than the two weighting factor calculation methods, such as the method of calculating a weighting factor duv(t) on the basis of the minimum value of the interregional phase coincidence degree cuv(t) and the method of calculating the weighting factor duv(t) in accordance with the distance between the two measurement regions ru(k) and rv(k), as described above; or the weighting factor duv(t) may be determined comprehensively by combining multiple methods.

### Third Embodiment

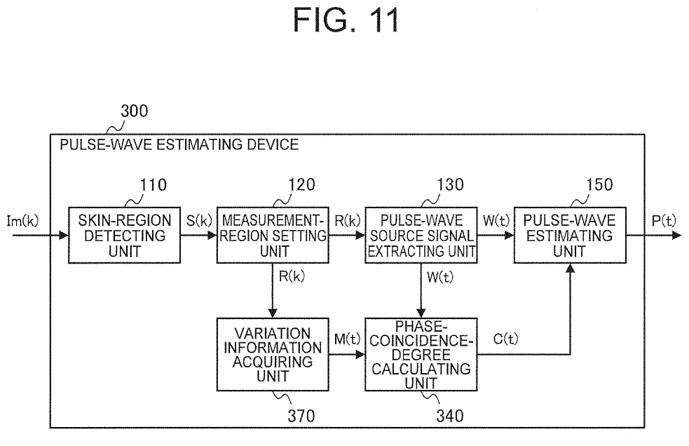

FIG. 11 is a block diagram schematically illustrating the configuration of a pulse-wave estimating device 300 serving as an information processing device according to the third embodiment.

The pulse-wave estimating device 300 is a device that can execute a pulse wave estimation method, which is an information processing method according to the third embodiment.

As illustrated in FIG. 11, the pulse-wave estimating device 300 includes a skin-region detecting unit 110, a measurement-region setting unit 120, a pulse-wave source signal extracting unit 130, a phase-coincidence-degree calculating unit 340, a pulse-wave estimating unit 150, and a variation information acquiring unit 370.

The skin-region detecting unit 110, the measurement-region setting unit 120, the pulse-wave source signal extracting unit 130, and the pulse-wave estimating unit 150 of the pulse-wave estimating device 300 according to the third embodiment are similar to the skin-region detecting unit 110, the measurement-region setting unit 120, the pulse-wave source signal extracting unit 130, and the pulse-wave estimating unit 150, respectively, of the pulse-wave estimating device 100 according to the first embodiment.

The variation information acquiring unit 370 specifies a variation in each measurement region ri(k) in measurement region information R(k) and generates variation information M(t) indicating the specified variation. The variation information M(t) is provided to the phase-coincidence-degree calculating unit 340.

The variation information M(t) can include element information mi(t) indicating movement on the image, a change in size, a change in shape, or the like of each measurement region ri(k).

The movement on the image is, for example, a two-dimensional vector indicating the difference between the position of a measurement region ri(e) in the e-th (where e is an integer greater than or equal to two) frame Im(e) on the image and the position of a corresponding measurement region ri(e−1) in the (e−1)-th frame Im(e−1) on the image.

As the positions of the measurement regions ri(e) and ri(e−1) on the image, for example, the centroid positions of the measurement regions ri(e) and ri(e−1) can be used. When a centroid position is used, the centroid coordinates of the four vertexes constituting each of the measurement regions ri(e) and ri(e−1) may be used as the centroid position.

The change in size is, for example, the difference between the area of the measurement region ri(e) in the e-th frame Im(e) and the area of the corresponding measurement region ri(e−1) in the (e−1)-th frame Im(e−1).

The change in shape is, for example, a four-dimensional vector indicating the difference between the ratio of the length of each side of the measurement region ri(e) in the e-th frame Im(e) to the total length of the four sides (there are four values because there are four sides) and the ratio of the length of each side of the corresponding measurement region ri(e−1) of the (k−1)-th frame Im(e−1) to the total length of the four sides.

The element information mi(t) is, for example, time-series data for Tp frames of the above-described information, and is extracted on the basis of, for example, measurement region information R(k−Tp), R(k−Tp+1) . . . , R(k) for the past Tp+1 frames.

For simplification purposes, in the following description, the element information mi(t) is assumed to be time-series data for Tp frames composed of two-dimensional vectors indicating the movement of the centroids of the respective measurement regions ri(k), and the variation information M(t) is assumed to be information that puts together the pieces of element information mi(t).

Note that the element information mi(t) does not have to be time-series data for Tp frames and may alternatively be composed of an arbitrary number of data pieces.

The phase-coincidence-degree calculating unit 340 according to the third embodiment, as in the second embodiment, selects multiple pairs each consisting of two pulse-wave source signals and calculates multiple coincidence degrees each indicating the degree of phase coincidence between corresponding base components in each of the pairs. The phase-coincidence-degree calculating unit 340 then sets weighting factors to the respective pairs, weights the coincidence degrees calculated for the respective pairs by using the weighting factors, and specifies the sums of the weighted values for the corresponding base components as multiple phase coincidence degrees.

In the third embodiment, the phase-coincidence-degree calculating unit 340 can set the weighting factors on the basis of the variation information M(t) so that the weights are heavier as the direction in which the two measurement regions ru(k) and rv(k) corresponding to each of the multiple pairs are disposed in a direction more similar to the direction in which the subject moves.

The phase-coincidence-degree calculating unit 340 can also set the weighting factors on the basis of the variation information M(t) so that, when the sizes of the two measurement regions ru(k) and rv(k) corresponding to each of the multiple pairs change in more similar ways, the weights are set heavier.

Moreover, the phase-coincidence-degree calculating unit 340 can also set weighting factors on the basis of the variation information M(t) so that, when the shapes of the two measurement regions ru(k) and rv(k) corresponding to each of the multiple pairs change in more similar ways, the weights are set heavier.

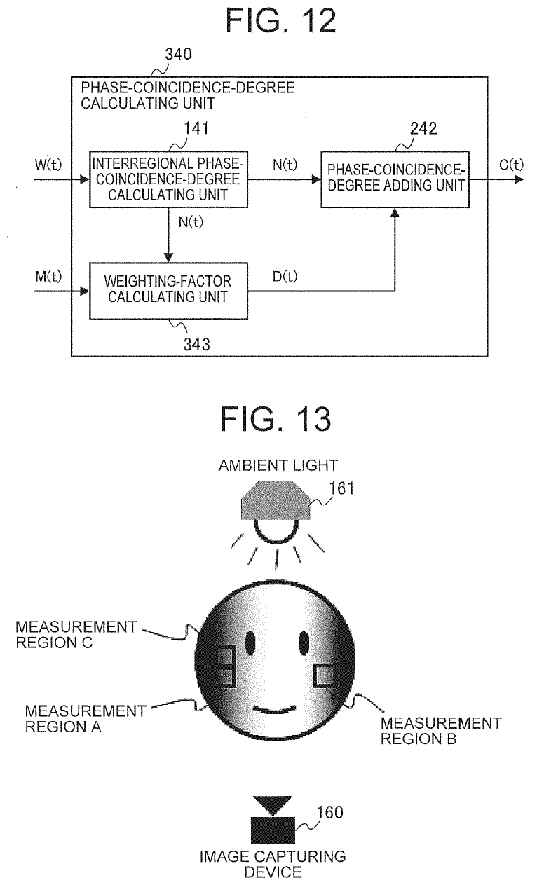

FIG. 12 is a block diagram schematically illustrating the configuration of the phase-coincidence-degree calculating unit 340.

The phase-coincidence-degree calculating unit 340 includes an interregional phase-coincidence-degree calculating unit 141, a phase-coincidence-degree adding unit 242, and a weighting-factor calculating unit 343.

The interregional phase-coincidence-degree calculating unit 141 of the phase-coincidence-degree calculating unit 340 according to the third embodiment is similar to the interregional phase-coincidence-degree calculating unit 141 of the phase-coincidence-degree calculating unit 140 according to the first embodiment. However, the interregional phase-coincidence-degree calculating unit 141 according to the third embodiment provides interregional phase-coincidence-degree information N(t) to the phase-coincidence-degree adding unit 242 and the weighting-factor calculating unit 343.

The phase-coincidence-degree adding unit 242 of the phase-coincidence-degree calculating unit 340 according to the third embodiment is similar to the phase-coincidence-degree adding unit 242 of the phase-coincidence-degree calculating unit 240 according to the second embodiment. However, the phase-coincidence-degree adding unit 242 according to the third embodiment obtains weighting information D(t) from the weighting-factor calculating unit 343.

The weighting-factor calculating unit 343 receives interregional phase-coincidence-degree information N(t) and variation information M(t), calculates weighting factors duv(t) for the respective interregional phase coincidence degrees cuv(t) indicated by the interregional phase-coincidence-degree information N(t) by using the variation information M(t), and generates weighting information D(t) indicating the calculated weighting factors duv(t). The weighting information D(t) is provided to the phase-coincidence-degree adding unit 242.

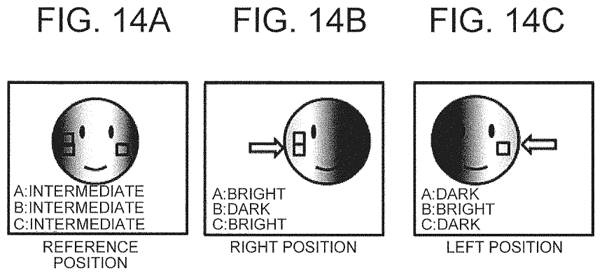

A method of calculating the weighting factors duv(t) on the basis of the variation information M(t) will be described with reference to FIGS. 13 and 14.

FIG. 13 is a schematic diagram illustrating the positional relationship of the face of the subject, an image capturing device 160, and a light source 161 of ambient light.

As illustrated in FIG. 13, it is assumed that a measurement region A, a measurement region B, and a measurement region C are disposed in the skin region of the face of the subject.

FIGS. 14A to 14C illustrate examples of images obtained by capturing the face of the subject by the image capturing device 160 illustrated in FIG. 13.

The image illustrated in FIG. 14A is an example in which the face of the subject is positioned at the center of the image capturing device 160. Such a position is referred to as a reference position.

The image illustrated in FIG. 14B is an example in which the face of the subject is positioned to the right of the center of the image capturing device 160. Such a position is referred to as a right position.

The image illustrated in FIG. 14C is an example in which the face of the subject is positioned to the left of the center of the image capturing device 160. Such a position is referred to as a left position.

When the face is positioned at the right position, the measurement region A and the measurement region C are brighter than when the face is positioned at the reference position; and when the face is positioned at the left position, the measurement regions A and C are darker than when the face is positioned at the reference position.

When the face is positioned at the right position, the measurement region B is darker than when the face is positioned at the reference position; and when the face is positioned at the left position, the measurement region B is brighter than when the face is positioned at the reference position.

As illustrated in FIGS. 14A to 14C, when the position of the face changes to the right or left, the luminance changes in different ways in measurement regions in a lateral positional relationship on the image, such as the measurement region A and the measurement region B.

In contrast, in measurement regions in a vertical positional relationship on the image, such as the measurement region A and the measurement region C, the luminance changes in similar ways.

That is, the luminance changes in different ways in measurement regions in a positional relationship in the same direction as the facial movement direction, and the luminance changes in similar ways in measurement regions in a positional relationship orthogonal with the facial movement direction.

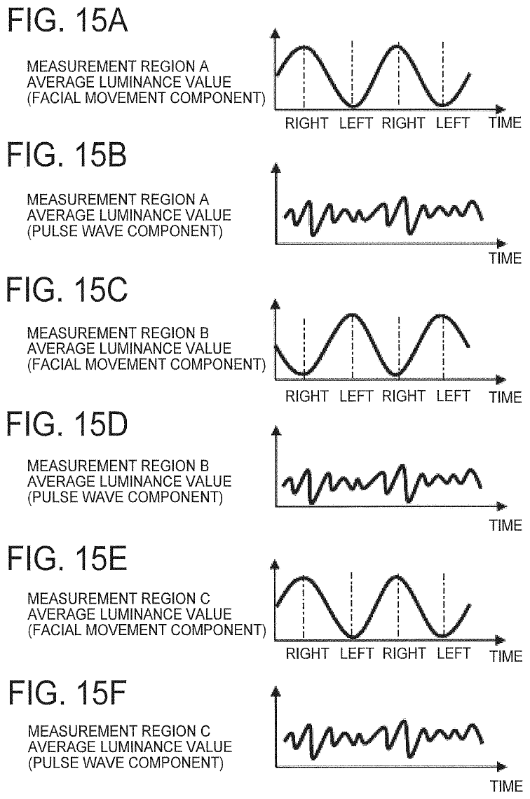

FIGS. 15A to 15F illustrate changes in the average luminance values in the measurement regions A, B, and C when the face of the subject moves in the order of the reference position, the right position, the reference position, the left position, the reference position, the right position, the reference position, and the left position.

FIG. 15A illustrates a change in the average luminance value of the facial movement component in the measurement region A when the face of the subject moves as described above; and FIG. 15B illustrates a change in the average luminance value of the pulse wave component of the face in the measurement region A when the face of the subject moves as described above.

FIG. 15C illustrates a change in the average luminance value of the facial movement components in the measurement region B when the face of the subject moves as described above; and FIG. 15D illustrates a change in the average luminance value of the pulse wave components of the face in the measurement region B when the face of the subject moves as described above.

FIG. 15E illustrates a change in the average luminance value of the facial movement components in the measurement region C when the face of the subject moves as described above; and FIG. 15F illustrates a change in the average luminance value of the pulse wave components of the face in the measurement region C when the face of the subject moves as described above.

As illustrated in FIGS. 15A and 15C, when the position of the face changes to the right or left, the phases of the facial movement components included in the average luminance value are different in the measurement regions (the measurement region A and the measurement region B) in a lateral positional relationship on the image.

As illustrated in FIGS. 15A and 15E, the phases of the facial movement components included in the average luminance value are different in the measurement regions (the measurement region A and the measurement region C) in a vertical positional relationship on the image.

Referring back to FIG. 12, the weighting-factor calculating unit 343 calculates a weighting factor duv(t) on the basis of the variation information M(t) by using the above-described features. Specifically, when the measurement regions ru(k) and rv(k) of a pair are in a positional relationship in the same direction as the two-dimensional vector included in the variation information M(t), the weighting factor duv(t) for the interregional phase coincidence degree cuv(t) is set to be large. In contrast, when the measurement regions ru(k) and rv(k) of a pair are in a vertical positional relationship, the weighting factor duv(t) is set to be small.

In the calculation of the weighting factor duv(t), the weighting-factor calculating unit 343 first specifies a representative vector Ms(t) (a two-dimensional vector) that is a representative motion vector included in the variation information M(t). For example, the weighting-factor calculating unit 343 may specify the maximum value of the two-dimensional vectors included in the variation information M(t) as the representative vector Ms(t).

In order to calculate the representative vector Ms(t), the weighting-factor calculating unit 343 first calculates an average value m_ave(t) (data having Tp two-dimensional vectors) of the element information mi(t) of the respective Rn measurement regions included in the variation information M(t). The weighting-factor calculating unit 343 then selects a two-dimensional vector having the largest vector length from the two-dimensional vectors included in the average value m_ave(t) and defines the selected vector as a selected vector M_max(t). The weighting-factor calculating unit 343 then converts the selected vector M_max(t) to a unit vector (a vector having a length of one) and defines the unit vector as the representative vector Ms(t).

Subsequently, the weighting-factor calculating unit 343 calculates a two-dimensional vector puv(t) indicating the relative positional relationship between the measurement regions corresponding to the respective interregional phase coincidence degrees cuv(t). The two-dimensional vector puv(t) is obtained by, for example, converting a two-dimensional vector puv_t(t) into a unit vector, where the two-dimensional vector puv_t(t) is obtained by calculating the difference between coordinate values of the two measurement regions ru(k) and rv(k), which are the source of the interregional phase coincidence degree cuv(t).

Finally, the weighting-factor calculating unit 343 calculates a weighting factor duv(s) from the two-dimensional vector puv(t) and the representative vector Ms(t). The weighting factor duv(t) is calculated as, for example, an absolute value of a dot product of the two-dimensional vector puv(t) and the representative vector Ms(t). Specifically, the weighting factor vector duv(t) is calculated by the following equation (3).

duv(t)=|puv(t)·Ms(t)| (3)

In equation (3), the symbol “·” represents the dot product of the vectors. The absolute value of the dot product of vectors having the same length approaches zero when the two vectors are in a substantially vertical relationship, whereas the absolute value is a large value in the positive direction when the two vectors are in a substantially parallel relationship. Since the two-dimensional vector puv(t) and the representative vector Ms(t) are both unit vectors having a length of one, the dot product of the vectors is substantially “0” when the vectors are in a substantially vertical relationship, whereas the dot product is substantially “1” when the vectors are in a substantially parallel relationship.

Therefore, according to equation (3), when the measurement regions ru(k) and rv(k) are in a positional relationship that is the same as the representative vector Ms(t), that is, when the representative vector Ms(t) and the two-dimensional vector puv(t) are substantially parallel to each other, the weighting factor duv(t) is large. In contrast, when the vectors are in a substantially vertical relationship, the weighting factor duv(t) is small.

The weighting-factor calculating unit 343 provides weighting information D(t) generated by putting together the weighting factors duv(t) calculated for the respective interregional phase coincidence degrees cuv(t) to the phase-coincidence-degree adding unit 242.

As described above, since the degree of phase misalignment of the movement components changes in accordance with the positional relationship between the facial movement direction and the measurement regions ri(k), the pulse-wave estimating device 300 according to the third embodiment can estimate a pulse wave from which movement components are further removed by calculating the weighting factor on the basis of the motion vectors of the measurement regions ri(k).

Note that, in the third embodiment, the element information mi(t) is two-dimensional vectors indicating the movement of the centroids of the respective measurement regions ri(k), but the element information mi(t) is not limited thereto. As described above, the movement of the four vertices of each measurement region ri(k), the change in the size or shape of each measurement region ri (k), or a combination of these may be used.

For example, the weighting-factor calculating unit 343 may set the weighting factor duv(t) to be large when the sizes of the measurement regions ru(k) and rv(k) included in the variation information M(t) change in similar ways and set the weighting factor duv(t) to be small when sizes do not change in similar ways. The sizes not changing in similar ways means that the respective measurement regions ru(k) and rv(k) are moving in different ways. Therefore, when the sizes do not change in similar ways, the degree of phase misalignment of the movement components is large, and it becomes easy to discriminate between the pulse wave components and the movement components.

Note that whether or not the sizes change in similar ways should be determined by using a similarity degree. The similarity degree is determined by using time-series data of the size change of the respective measurement regions ru(k) and rv(k). For example, when the size of a certain frame is “1,” the weighting-factor calculating unit 343 specifies how the size of the measurement region in the subsequent frame has transitioned. For example, the weighting-factor calculating unit 343 specifies time-series data indicating that the transition from the first frame to the fifth frame is “1,” “0.9,” “0.8,” “0.8,” and “0.9.”

The weighting-factor calculating unit 343 then specifies the time-series data in the respective measurement regions ri(k) and calculates a correlation value of the time-series data in pairs of measurement regions ru(k) and rv(k). The correlation value is a value from “1” to “−1,” where “1” indicates similar changes and “−1” indicates changes that are not similar. Since the weighting factor duv(t) is preferably set large when the correlation value is small and set small when the correlation value is large, the weighting-factor calculating unit 343 calculates the weighting factor duv(t) by using, for example, the following equation (4):

Weighting factor duv(t)=1−(correlation value between two measurement regions ru(k) and rv(k)) (4)

The weighting-factor calculating unit 343 may set the weighting factor duv(t) to be large when the shapes of the measurement regions ru(k) and rv(k) included in the variation information M(t) change in similar ways and set the weighting factor duv(t) to be small when the shapes do not change in similar ways. Whether or not the shapes change in similar ways is determined by similarity, and the similarity is determined by using time-series data of the change in a four-dimensional vector indicating the change in the shapes of the measurement regions ru(k) and rv(k).

For example, the weighting-factor calculating unit 343 calculates correlation values from the respective elements of the respective four-dimensional vectors indicating the changes in the shapes of the respective measurement regions ru(k) and rv(k). Since four correlation values are calculated here, the weighting-factor calculating unit 343 calculates the weighting factor duv(t) by the following equation (5) by using the average of the four calculated correlation values.

Weighting factor duv(t)=1−(average correlation value of two measurement regions ru(k) and rv(k)) (5)

Note that the weighting-factor calculating unit 343 may use, for example, the average of “a weighting factor calculated on the basis of a change in size” and “a weighting factor calculated on the basis of a change in shape” as a final weighting factor duv(t) for the pair of measurement regions ru(k) and rv(k).

### Fourth Embodiment

As illustrated in FIG. 1, a pulse-wave estimating device 400 according to the fourth embodiment includes a skin-region detecting unit 110, a measurement-region setting unit 120, a pulse-wave source signal extracting unit 130, a phase-coincidence-degree calculating unit 440, and a pulse-wave estimating unit 150.

The skin-region detecting unit 110, the measurement-region setting unit 120, the pulse-wave source signal extracting unit 130, and the pulse-wave estimating unit 150 of the pulse-wave estimating device 400 according to the fourth embodiment are similar to the skin-region detecting unit 110, the measurement-region setting unit 120, the pulse-wave source signal extracting unit 130, and the pulse-wave estimating unit 150, respectively, of the pulse-wave estimating device 100 according to the first embodiment.

Note that the pulse-wave estimating device 400 is a device that can execute a pulse wave estimating method that is an information processing method according to the fourth embodiment.

The phase-coincidence-degree calculating unit 440 according to the fourth embodiment selects multiple pairs each consisting of two pulse-wave source signals from the multiple pulse-wave source signals indicated by pulse-wave source signal information W(t). The phase-coincidence-degree calculating unit 440 then calculates multiple coincidence degrees each indicating the degree of phase coincidence between corresponding base components in the respective selected pairs, as in the first embodiment. The phase-coincidence-degree calculating unit 440 then sets weighting factors to the respective pairs, weights the multiple coincidence degrees calculated for the respective pairs by using the weighting factors, and specifies the sums of the weighted values for the respective corresponding base components as phase coincidence degrees.

The phase-coincidence-degree calculating unit 440 can set the weighting factors so that the weights are large for the coincidence degrees corresponding to the measurement regions having high degrees of phase coincidence out of the multiple coincidence degrees calculated from respective pairs. The weighting factors of the coincidence degrees corresponding to the measurement regions ri can be set in accordance with the magnitude of the amplitudes of the pulse-wave source signals wi(t).

As illustrated in FIG. 10, the phase-coincidence-degree calculating unit 440 of the fourth embodiment includes an interregional phase-coincidence-degree calculating unit 141, a phase-coincidence-degree adding unit 242, and a weighting-factor calculating unit 443.

The interregional phase-coincidence-degree calculating unit 141 of the phase-coincidence-degree calculating unit 440 of the fourth embodiment is similar to the interregional phase-coincidence-degree calculating unit 141 of the phase-coincidence-degree calculating unit 140 of the first embodiment. However, the interregional phase-coincidence-degree calculating unit 141 of the fourth embodiment provides interregional phase-coincidence-degree information N(t) to the phase-coincidence-degree adding unit 242 and the weighting-factor calculating unit 443.

The weighting-factor calculating unit 443 receives the interregional phase-coincidence-degree information N(t), calculates weighting factors duv(t) for the respective interregional phase coincidence degrees cuv(t) indicated by the interregional phase-coincidence-degree information N(t), and generates weighting information D(t) indicating the calculated weighting factors duv(t). The weighting information D(t) is provided to the phase-coincidence-degree adding unit 242.

The weighting information D(t) can include the weighting factors duv(t) for the respective interregional phase coincidence degrees cuv(t) included in the interregional phase-coincidence-degree information N(t). A weighting factor duv(t) is, for example, a value between “0” and “1.” For example, when the weighting factor duv(t) is “0,” the weight for the corresponding interregional phase coincidence degree cuv(t) is small, and when the weighting factor duv(t) is “1,” the weight for the corresponding interregional phase coincidence degree cuv(t) is large. When the weighting factor duv(t) is “0.5,” the weight is an intermediate value between those of “0” and “1.”

The weighting factor duv(t) is determined on the basis of, for example, a representative value eu(t) of an interregional phase coincidence degree cui(t) associated with a measurement region ru and a representative value ev(t) of an interregional phase coincidence degree cvi(t) associated with a measurement region ry (where i=1, 2 . . . , Rn). The representative value eu(t) of the interregional phase coincidence degree associated with the measurement region ru is, for example, the average of the interregional phase coincidence degrees cui(t) associated with the measurement region ru. Specifically, the average of the degrees of phase coincidence for the respective frequency components calculated for the interregional phase coincidence degrees cu1 (t), cu2 (t) . . . , cuRn (t) is defined as the representative value eu(t) of the interregional phase coincidence degrees associated with the measurement region ru. When the components of the pulse wave signals included in the pulse-wave source signal wu(t) of the measurement region ru are strong, the degree of phase coincidence is high for any frequency component of an interregional phase coincidence degree cui associated with the measurement region ru. In contrast, when the components of the pulse wave signals included in the pulse-wave source signal wu(t) of the measurement region ru are weak, the degree of phase coincidence is low for any frequency component of an interregional phase coincidence degree cui associated with the measurement region ru. That is, when the representative value eu(t) associated with the measurement region ru is large, the weighting factor dui corresponding to the measurement region ru is set large, and when the representative value eu(t) is small, the weighting factor dui corresponding to the measurement region ru is set small, to weight the measurement region having strong components of a pulse wave signal, i.e., components having high phase coincident with another measurement regions.

The weighting factor duv(t) is calculated from the representative value eu(t) of the interregional phase coincidence degrees associated with the measurement region ru and the representative value ev(t) of the interregional phase coincidence degrees associated with the measurement region ru.

In calculating the weighting factor duv(t), first, the weighting factor du(t) for the measurement region ru and the weighting factor dv(t) for the measurement region ry are calculated. Since the calculation methods of the weighting factors du(t) and dv(t) are similar, the calculation method of the weighting factor du(t) will be described here.

The maximum value and the minimum value that can be taken by the representative value eu(t) of the interregional phase coincidence degrees are eₘₐₓ and eₘᵢₙ, respectively. The weighting factor du(t) is calculated by the following equation (6). The maximum value eₘₐₓ and the minimum value eₘᵢₙ, are assumed to be predetermined.

du(t)=1.0−(eu(t))/(eₘₐₓ−eₘᵢₙ) (6)

After the weighting factor dv(t) has been calculated in a similar manner, the weighting factor duv(t) is calculated as the minimum value of the weighting factors du(t) and dv(t). That is, duv(t)=min(du(t), dv(t)).

By calculating the weighting factor duv(t) using the above equation, the weighting factor associated with the measurement region having strong components of the pulse wave signals, i.e., components having phases coinciding with those of other measurement regions can be set to a large value.

As described above, the phase matching degree calculating unit 440 of the pulse-wave estimating device 400 according to the fourth embodiment uses multiple coincidence degrees calculated from the respective pairs to calculate representative values of the two measurement regions included in each of the pairs, and sets weighting factors so that the larger the representative values, the heavier the weights. Note that the representative value of each of the measurement regions for the multiple coincidence degrees calculated from the respective pairs is, for example, the representative value eu(t) of the interregional phase coincidence degrees cui(t) associated with the measurement region ru, or the representative value ev(t) of the interregional phase coincidence degrees cvi(t) associated with the measurement region rv. Therefore, the fourth embodiment can estimate a pulse with higher accuracy.

The weighting-factor calculating unit 443 puts together the weighting factors duv(t) calculated for the respective interregional phase coincidence degrees cuv(t) and generates weighting information D(t) indicating the weighting factors duv(t). The weighting information D(t) is provided to the phase-coincidence-degree adding unit 242.

The phase-coincidence-degree adding unit 242 receives the interregional phase-coincidence-degree information N(t) and the weight information D(t), generates phase coincidence degree information C(t) for every base component between the measurement regions and provides the generated phase coincidence degree information C(t) to the pulse-wave estimating unit 150.

The phase-coincidence-degree adding unit 242 multiplies the interregional phase coincidence degrees cuv(t) indicated in the interregional phase-coincidence-degree information N(t) by the corresponding weight information D(t) and adds the results for respective base components to generate phase coincidence degree information C(t) indicating the degrees of phase coincidence between the measurement regions ri(k) for all base components. The phase coincidence degree information C(t) is provided to the pulse-wave estimating unit 150.

The pulse-wave estimating unit 150 estimates a pulse wave on the basis of the pulse-wave source signal information W(t) and the phase coincidence degree information C(t) and outputs a pulse-wave estimation result P(t) that is pulse wave information indicating the estimated pulse wave. For example, when the pulse rate is output as the pulse-wave estimation result P(t), the pulse-wave estimating unit 150 specifies the frequency component that is the base component having the highest degree of phase coincidence in the phase coincidence degree information C(t) for every frequency component and estimates the pulse wave on the basis of the specified frequency component. Specifically, the pulse-wave estimating unit 150 assumes that the frequency component having the highest degree of phase coincidence corresponds to the pulse wave and outputs the frequency of the frequency component corresponding to the pulse wave as the pulse rate.

Note that, in the fourth embodiment, the representative value eu(t) of the interregional phase coincidence degrees associated with the measurement region ru is the average of the interregional phase coincidence degrees cui(t) associated with the measurement region ru, but the representative value eu(t) is not limited thereto. For example, the representative value eu(t) may be the median or minimum value or may be the number of times that the degree of phase coincidence exceeds a threshold value for each frequency component.

Note that, in the fourth embodiment, the value of the weight for each measurement region is determined on the basis of only the interregional phase coincidence degree, but the determination of the weight is not limited thereto. For example, as described in Non-patent Literature 1, the weighting factor for each measurement region may be calculated on the basis of the difference between the maximum value and the minimum value of the pulse-wave source signal wi(t) or the signal-noise ratio (SNR) of a power spectrum or may be calculated on the basis of a combination of these. As a combining method, for example, the average of the weighting factors calculated by the SNR of the power spectrum and the weighting factor calculated on the basis of the interregional phase coincidence degree for a measurement region ru is defined as a weighting factor du(t) for the measurement region.

In the fourth embodiment, it is assumed that the frequency component having the highest degree of phase coincidence corresponds to a pulse wave, and the frequency of the frequency component corresponding to the pulse wave is output as a pulse rate; however, even when the phases coincide, if the amplitude of the signal is larger or smaller than expected, the frequency component having the highest degree of phase coincidence may be output as the pulse rate after the corresponding frequency component has been removed.

The amplitude of the frequency component corresponding to the pulse wave changes in accordance with the brightness (luminance value) of the skin region in frames Im(t) or the tone, thickness, or blood flow rate of the skin of the subject. Among these, the brightness of the skin region in the frames Im(t) has a large influence, and the amplitude of the frequency component corresponding to the pulse wave can be estimated on the basis of the brightness of the skin region. For example, thresholds θH(Iₐᵥₑ(t)) and θL(Iₐᵥₑ(t)) for the amplitude of the frequency component determined on the basis of the average luminance value Iₐᵥₑ(t) of all measurement regions are used to specify the frequency component having the highest degree of phase coincidence for only the frequency components of which the amplitude is within the range of the threshold θH to the threshold θL, inclusive.

In this way, by estimating the pulse rate from the frequency component having an amplitude within a predetermined range, the pulse rate can be estimated with higher accuracy.

### DESCRIPTION OF REFERENCE CHARACTERS

100, 200, 300, 400 pulse-wave estimating device; 110 skin-region detecting unit; 120 measurement-region setting unit; 130 pulse-wave source signal extracting unit; 140, 240, 340, 440 phase-coincidence-degree calculating unit; 141 interregional phase-coincidence-degree calculating unit; 142, 242 phase-coincidence-degree adding unit; 243, 343, 443 weighting-factor calculating unit; 150 pulse-wave estimating unit; 160 image capturing device; 161 light source; 370 variation-information acquiring unit.

1. An information processing device comprising processing circuitry
to detect a skin region in each of multiple frames representing video footage in a predetermined time period, the skin region including skin of a person;
to set multiple measurement regions in the skin region;
to extract, from the multiple measurement regions, multiple pulse-wave source signals corresponding to the respective measurement regions, each of the extracted pulse-wave source signals indicating a change in luminance in the predetermined time period;
to calculate multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and
to specify one of the phase coincidence degrees having the highest degree of phase coincidence of the multiple phase coincidence degrees and estimate a pulse wave of the person based on the base components corresponding to the specified phase coincidence degree, wherein
the processing circuitry selects multiple pairs each consisting of a first pulse-wave source signal and a second pulse-wave source signal selected from the multiple pulse-wave source signals, calculates interregional phase coincidence degrees between the multiple base components constituting the first pulse-wave source signal and the multiple base components constituting the second pulse-wave source signal in each of the multiple pairs to calculate the interregional phase coincidence degrees corresponding to the respective base components, sets weighting factors based on at least one of the interregional phase coincidence degrees calculated for the respective multiple pair, magnitudes of the base components, disposition of the measurement regions, size of the measurement regions, and shapes of the measurement regions, and calculates the multiple phase coincidence degrees by applying weights by using the weighting factors and adding the multiple interregional phase coincidence degrees calculated in the respective pairs, each of the multiple interregional phase coincidence degrees being added for each of the corresponding base components.

2.-4. (canceled)

5. The information processing device according to claim 1, wherein the processing circuitry sets the weighting factors such that the higher in the coincidence degrees between the multiple base components constituting the first pulse-wave source signal and the multiple base components constituting the second pulse-wave source signal for each of the base components the multiple interregional phase coincidence degrees calculated for the respective pairs are, the heavier the weights are.

6. The information processing device according to claim 1, wherein the processing circuitry sets the weighting factors such that the larger the distance between two of the measurement regions corresponding to each of the multiple pairs is, the heavier the weights are.

7. The information processing device according to claim 1, wherein the processing circuitry sets the weighting factors such that the more similar the direction in which two of the measurement regions corresponding to each other in each of the multiple pairs are arranged is to the direction of the movement of the person is, the heavier the weights are.

8. The information processing device according to claim 1, wherein the processing circuitry sets the weighting factors such that the more similar the changes in the sizes of two of the measurement regions corresponding to each other in each of the multiple pairs are, the heavier the weights are.

9. The information processing device according to claim 1, wherein the processing circuitry sets the weighting factors such that the more similar the changes in the shapes of two of the measurement regions corresponding to each other in each of the multiple pairs are, the heavier the weights are.

10. The information processing device according to claim 1, wherein the processing circuitry calculates a representative value of two of the measurement regions included in each of the multiple pairs by using the multiple interregional phase coincidence degrees calculated for the respective pairs, and sets the weighting factors such that the higher the representative value is, the heavier the weight is.

11. The information processing device according to claim 1, wherein the base components are frequency components of the pulse-wave source signals.

12. The information processing device according to claim 1, wherein,
the base components are frequency components of the pulse-wave source signals, and
the interregional phase coincidence degrees are each an absolute value of a phase difference between one of the frequency components constituting the first pulse-wave source signal and a corresponding one of the corresponding frequency components constituting the second pulse-wave source signal.

13. A non-transitory computer-readable medium that stores therein a program that causes a computer to execute processes of:
detecting a skin region in each of multiple frames representing video footage in a predetermined time period, the skin region including skin of a person;
setting multiple measurement regions in the skin region;
extracting, from the multiple measurement regions, multiple pulse-wave source signals corresponding to the respective measurement regions, each of the extracted pulse-wave source signals indicating a change in luminance in the predetermined time period;
calculating multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and
specifying one of the phase coincidence degrees having the highest degree of phase coincidence of the multiple phase coincidence degrees and estimate a pulse wave of the person based on the base components corresponding to the specified phase coincidence degree, wherein
when the multiple phase coincidence degrees are calculated, multiple pairs each consisting of a first pulse-wave source signal and a second pulse-wave source signal selected from the multiple pulse-wave source signals are selected, interregional phase coincidence degrees between the multiple base components constituting the first pulse-wave source signal and the multiple base components constituting the second pulse-wave source signal in each of the multiple pairs are calculated to calculate the interregional phase coincidence degrees corresponding to the respective base components, weighting factors are set based on at least one of the interregional phase coincidence degrees calculated for the respective multiple pair, magnitudes of the base components, disposition of the measurement regions, size of the measurement regions, and shapes of the measurement regions, and the multiple phase coincidence degrees are calculated by applying weights by using the weighting factors and adding the multiple interregional phase coincidence degrees calculated in the respective pairs, each of the multiple interregional phase coincidence degrees being added for each of the corresponding base components.

14. An information processing method comprising:
detecting a skin region in each of multiple frames representing video footage in a predetermined time period, the skin region including skin of a person;
setting multiple measurement regions in the skin region;
extracting, from the multiple measurement regions, multiple pulse-wave source signals corresponding to the respective measurement regions, each of the extracted pulse-wave source signals indicating a change in luminance in the predetermined time period;
calculating multiple phase coincidence degrees corresponding to the respective pulse-wave source signals, each of the phase coincidence degrees indicating a degree of phase coincidence between phases of corresponding base components of the respective pulse-wave source signals, each of the pulse-wave source signals including a plurality of the base components; and
specifying one of the phase coincidence degrees having the highest degree of phase coincidence of the multiple phase coincidence degrees and estimate a pulse wave of the person based on the base components corresponding to the specified phase coincidence degree, wherein
when the multiple phase coincidence degrees are calculated, multiple pairs each consisting of a first pulse-wave source signal and a second pulse-wave source signal selected from the multiple pulse-wave source signals are selected, interregional phase coincidence degrees between the multiple base components constituting the first pulse-wave source signal and the multiple base components constituting the second pulse-wave source signal in each of the multiple pairs are calculated to calculate the interregional phase coincidence degrees corresponding to the respective base components, weighting factors are set based on at least one of the interregional phase coincidence degrees calculated for the respective multiple pair, magnitudes of the base components, disposition of the measurement regions, size of the measurement regions, and shapes of the measurement regions, and the multiple phase coincidence degrees are calculated by applying weights by using the weighting factors and adding the multiple interregional phase coincidence degrees calculated in the respective pairs, each of the multiple interregional phase coincidence degrees being added for each of the corresponding base components.
